(19) United States
(12) Patent Application Publication
(10) Pub. No.: US 2023/0372364 A1
(43) Pub. Date: Nov. 23, 2023

(54) METHOD OF TREATING GABA MEDIATED DISORDERS

(71) Applicants: Jeremy HILL, Boston, MA (US); Edward MONAGHAN, Boston, MA (US); Amy CHAPPELL, Boston, MA (US); Eliem Therapeutics (UK) Ltd, Altrincham (GB)

(72) Inventors: Jeremy Hill, Boston, MA (US); Edward Monaghan, Boston, MA (US); Amy Chappell, Boston, MA (US)

(21) Appl. No.: 18/027,752

(22) PCT Filed: Sep. 30, 2021
(86) PCT No.: PCT/US2021/052859

§ 371 Date: Mar. 22, 2023

Related U.S. Application Data
(60) Provisional application No. 63/163,214, filed on Mar. 19, 2021.
(60) Provisional application No. 63/086,440, filed on Oct. 1, 2020.

(51) Int. Cl. A61K 31/58; A61P 25/24; A61P 25/22; A61P 25/08

(52) U.S. Cl. CPC A61K 31/58; A61P 25/24; A61P 25/22; A61P 25/08

(57) ABSTRACT

The invention generally relates to methods of treating a central nervous system related disorder in a human subject, the method comprising providing a pharmaceutical composition to a human subject having a central nervous system disorder, wherein the pharmaceutical composition is an oral formulation that comprises a compound of Formula (I): at a dose between 5 mg and 90 mg.

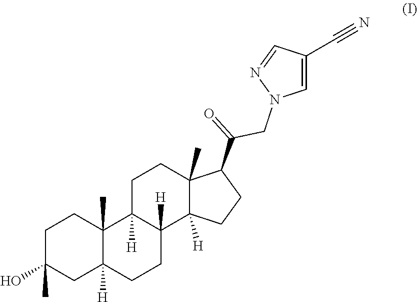

### FIELD OF THE INVENTION

The invention relates to methods of treating central nervous system related disorders.

### BACKGROUND

Psychiatric illnesses are a major cause of disability and death globally. Many of these disorders are central nervous system disorders that debilitate the ability for sufferers to function. Moreover, individuals suffering from severe central nervous system disorders are also at increased risk of death from metabolic and cardiovascular illness. Current treatments, including medications, neuromodulation methods, and psychotherapy, can be effective but many individuals fail to respond to treatment. Even among those who respond, relapses are common.

The GABA_{A} receptor is a receptor with wide distribution in the central nervous system of mammals and plays a role in virtually all brain functions. The GABA_{A} receptor is responsible for most of the physiological activity of GABA in the central nervous system, a major inhibitory neurotransmitter. GABA_{A} receptor positive allosteric modulators are a broad class of compounds for mediating GABA_{A} receptor activity that includes benzodiazepines, barbiturates, and neuroactive steroids.

Neuroactive steroids are unique among GABA_{A} receptor positive allosteric modulators in their binding profile and physiological effect. Neuroactive steroids have also been shown to have antiepileptic, anxiolytic, and antidepressant properties in both humans and animals. For example, other neuroactive steroids that are approved or in development include brexanolone, sold under the trademark ZULRESSO by Sage Therapeutics and approved for the treatment of postpartum depression, zuranolone, labeled SAGE-217 by Sage Therapeutics and in development for the treatment of major depressive disorders, and ganaxalone, in development by Marinus Pharmaceuticals for the treatment of rare epilepsies and postpartum depression.

However, other neuroactive steroids have little or no intrinsic activity at GABA_{A} receptors and yet others inhibit the action of neuroactive steroids that do act as positive allosteric modulators. Moreover, the side-effects, abuse potential, and interaction with other medications from neuroactive steroids remains not fully understood. Given the ongoing suffering of individuals with disorders of the central nervous system, there is substantial need to develop new treatments for these disorders

### SUMMARY

The invention provides methods of treating a central nervous system related disorder in a human subject by providing a pharmaceutical composition comprising a compound having the structure of Formula (I):

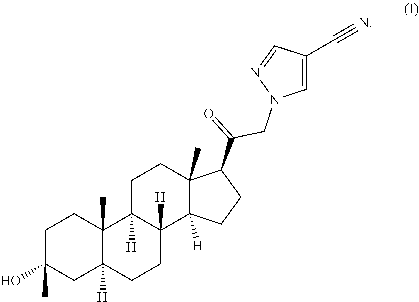

The compound of Formula (I) is a novel neuroactive steroid that acts as a positive allosteric modulator of receptors for the neurotransmitter γ-aminobutyric acid (GABA), for example the GABA_{A} receptor. The invention recognizes that various central nervous system related disorders, such as seizure disorders and mood disorders, can be ameliorated by providing the compound of Formula (I). Therefore, the invention provides methods of treating central nervous system related disorders using compositions that contain therapeutically effective amounts of the compound of Formula (I).

More specifically, the invention provides doses and dosage regimens for pharmaceutical compositions that include Formula (I) that are safe and tolerable for administration to humans. Moreover, the pharmacokinetic profile of the compound of Formula (I) has been found to be suitable for administration to human subjects. Accordingly, it has been discovered by the present invention that a pharmaceutical composition including a compound of Formula (I) administered at doses between 5 mg and 90 mg is a safe, tolerable, and effective for treatment for a variety of CNS indications in humans, including seizure disorders, mood disorders, and related conditions.

In that manner, the invention provides methods of treating a central nervous system related disorder in a human subject, the method comprising providing a pharmaceutical composition to a human subject having a central nervous system disorder, wherein the pharmaceutical composition is an oral formulation that comprises a compound of Formula (I):

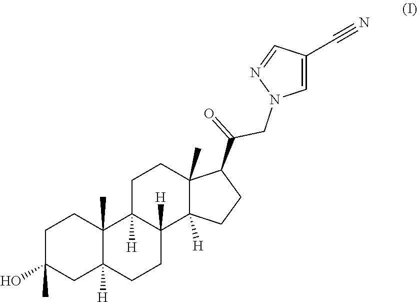

at a dose between 5 mg and 90 mg.

The pharmaceutical composition may be provided daily. In aspects of the invention, the pharmaceutical composition is provided once every seven days. In aspects of the present invention, the pharmaceutical composition may be provided for at least seven days, for at least fourteen days, for at least one month, for at least two months, for at least three months, for at least six months, or for at least one year.

In aspects of the invention, the pharmaceutical composition is provided to the subject in the morning. The pharmaceutical composition may be provided to the subject in the evening. The pharmaceutical composition may be provided to the subject with food. The pharmaceutical composition may be provided after fasting.

The composition may be provided as 5 mg tablets, 10 mg tablets, 15 mg, tablets, 20 mg tablets, 30 mg tablets, 40 mg tablets, 50 mg tablets, 100 mg tablets or at any amount in the between 5 mg and 100 mg of the compound of Formula (I). For example, where a dose of 90 mg is provided to a subject, the dose may be provided as three 30 mg tablets.

In other aspects of the invention, the pharmaceutical composition may be provided as multiple doses per day. The pharmaceutical composition may be provided in two, three, four, five, six, or more doses per day. The pharmaceutical composition may be provided at a dose of about 5 mg to about 90 mg. The pharmaceutical composition may be provided at a dose between about 5 mg and about 1000 mg. For example, the pharmaceutical composition may be provided at a dose of 5 mg, 15 mg, 30 mg, 60 mg, 90 mg, 100 mg, 125 mg, 150 mg, 175 mg, 200 mg, 250 mg, 300 mg, 350 mg, 400 mg, 500 mg, 600 mg, 700 mg, 800 mg, or 1000 mg. The pharmaceutical composition may be provided in the composition at the maximum tolerated dose.

The pharmaceutical composition administered at doses disclosed by the present invention is a safe, tolerable, and effective treatment for a variety of CNS indications in humans, including seizure disorders, mood disorders, and related conditions. Accordingly, the methods of the invention result in minimal serious adverse reactions or no serious adverse reactions related to administration of the composition. An adverse event of reaction is an untoward medical occurrence that may be related or may not be related to administration of the composition. An adverse reaction is considered to be related to administration of the composition if a temporal relationship between the onset of the adverse reaction relative to the administration of the composition is reasonable, follows a known response pattern to the treatment, and an alternative cause is unlikely. Minimal adverse reactions means fewer than two identical severe adverse reactions over a seven day period or fewer than three severe adverse reactions deemed related to administration of the composition over a seven day period. A serious adverse reaction is a reaction that causes severe discomfort, and daily activities are significantly impaired or prevented.

The methods of the invention also may result in no serious adverse events. A serious adverse event or reaction is any untoward medical occurrence that: (i) results in death; (ii) is life-threatening; (iii) requires in-patient hospitalization or prolongation of existing hospitalization; (iv) results in persistence or significant disability/incapacity; (v) is a congenital anomaly/birth defect.

The disorder may be any disease, disorder, or condition caused by or associated with GABA and/or GABA_{A} receptor activity. For example, the condition may be selected from the group consisting of epilepsy, sleep disorders, spasticity, anxiety disorders, schizophrenia, stiff-person syndrome, premenstrual dysphoric disorder, depressive disorders, such as postpartum depression, seizure disorders, and mood disorders, The condition may be selected from the group consisting of seizure disorders and mood disorder.

In another aspect, the invention provides uses of a compound of Formula (I) for making a medicament for treating a subject suffering from a central nervous system disorder according to the dosing regimen described above. The medicament may be for treatment of one of the conditions described above.

In another aspect, the invention provides use of a compound of Formula (I) for the treatment of a subject suffering from a central nervous system disorder according to the dosing regimen described above. The use may be for treatment of one of the conditions described above.

### BRIEF DESCRIPTION OF THE DRAWINGS

FIG. 1 is a flow chart of a single ascending dose study design of the compound of Formula (I).

FIG. 2 is a flow chart of a multiple ascending dose study design of the compound of Formula (I).

FIG. 3 depicts Table 1, a schedule of activities for a single active dose study of the compound of Formula (I).

FIG. 4 depicts Table 2, a schedule of activities for a multiple active dose study of the compound of Formula (I).

FIG. 5 is a graph of average plasma concentration of CV-10155 at various time points following oral administration to dogs.

FIG. 6 is a graph of average plasma concentration of CV-10155 at various time points following oral administration to dogs.

FIG. 7 is a graph of plasma concentration of CV-10155 in individual dogs at various time points following oral administration of 1 mg/kg CV-10155 in a formulation containing labrasol.

FIG. 8 is a graph of plasma concentration of CV-10155 in individual dogs at various time points following oral administration of 1 mg/kg CV-10155 in a formulation containing labrasol/capryol 80:20.

FIG. 9 is a graph of plasma concentration of CV-10155 in individual dogs at various time points following oral administration of 1 mg/kg CV-10155 in a formulation containing 30% 2-hydroxypropyl-beta-cyclodextrin (HPbCD).

FIG. 10 is a graph of plasma concentration of CV-10155 in individual dogs at various time points following intravenous administration of 1 mg/kg CV-10155 in a formulation containing 30% HPbCD.

FIG. 11 is a graph of plasma concentration of CV-10155 in individual dogs at various time points following oral administration of 1 mg/kg CV-10155 in a formulation containing 1:4 HPMC-AS-MG.

FIG. 12 is a graph of plasma concentration of CV-10155 in individual dogs at various time points following oral administration of 1 mg/kg CV-10155 in a formulation containing 1:4 HPMC-E3.

FIG. 13 is a graph of plasma concentration of CV-10155 in individual dogs at various time points following oral administration of 1 mg/kg CV-10155 in a formulation containing 1:4 PVP VA64.

FIG. 14 is a graph of plasma concentration of CV-10155 in individual dogs at various time points following oral administration of 1 mg/kg CV-10155 in a formulation containing 1:4 Eudragit L100-55.

FIG. 15 is a graph of the average plasma concentration of CV-10155 following oral administration to humans.

FIG. 16 is a graph of the average plasma concentration of CV-10155 following oral administration of a 30 mg dose to humans.

FIG. 17 is a graph showing the ratios of Cₘₐₓ and AUC between fed and fasted subjects.

FIG. 18 is a graph of the average plasma concentration of CV-10155 following oral administration to humans.

FIG. 19 is a graph of Cₘₐₓ ranges from studies on rats, dogs, and humans.

FIG. 20 is a graph of AUC₀₋₂₄ ranges from studies on rats, dogs, and humans. B FIG. 21 is a hypnogram showing the percentage of time spent in different sleep states by rats given drug vehicle.

FIG. 22 is a hypnogram showing the percentage of time spent in different sleep states by rats given 1 mg/kg CV-10155.

FIG. 23 is a hypnogram showing the percentage of time spent in different sleep states by rats given 3 mg/kg CV-10155.

FIG. 24 is a hypnogram showing the percentage of time spent in different sleep states by rats given 6 mg/kg CV-10155.

FIG. 25 is a hypnogram showing the percentage of time spent in different sleep states by rats given 10 mg/kg CV-10155.

### DETAILED DESCRIPTION

The present invention provides methods of treating a central nervous system related disorder, the method comprising providing a pharmaceutical composition to a subject having a central nervous system disorder, the pharmaceutical composition including a compound of Formula (I):

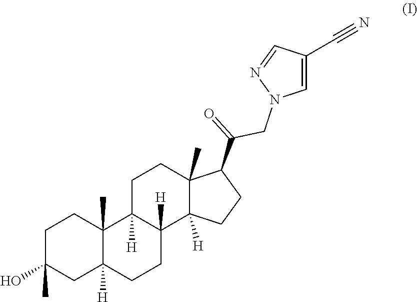

wherein the compound of Formula (I) is provided orally at a dose between 5 mg and 90 mg. In aspects of the invention, the compound of Formula (I) is provided at a dose of 5 mg, 15 mg, 30 mg, 60 mg, or 90 mg.

The compound of Formula (I), referred to as ATN-10155, has the IUPAC name 1-[2-[(3R,5S,8R,9S,10S,13S,14S,17S)-3-hydroxy-3,10,13-trimethyl-1,2,4,5,6,7,8,9,11,12,14,15,16,17-tetradecahydrocyclopenta[a]phenanthren-17-yl]-2-oxoethyl]pyrazole-4-carbonitrile and is disclosed in International Patent Publication No. WO 2016/061527, the contents of which are incorporated herein by reference.

The compound of Formula (I) is an allosteric modulator of receptors for the neurotransmitter γ-aminobutyric acid (GABA), for example the GABA_{A} receptor. The invention recognizes that various central nervous system related disorders, such as seizure disorders and mood disorders, can be ameliorated by providing the compound of Formula (I). Therefore, the invention provides methods of treating central nervous system related disorders using compositions that contain therapeutically effective amounts of the compound of Formula (I).

Without wishing to be bound by theory or mechanisms of action, it is believed that the compound of Formula (I) is a GABA_{A} receptor positive allosteric modulator. It is believed that providing the compound of Formula (I) may overcome fluctuations and stabilize GABA_{A} receptor signaling. For example, the compound of Formula (I) may be provided in amounts to GABA_{A} receptors to control signaling of such receptors. In that manner, the compound of Formula (I) may provide stimulation to the GABA_{A} receptor. Stabilization of GABA_{A} receptor signaling may be achieved by providing a continuous dose of the compound of Formula (I). Alternatively, GABA_{A} receptor signaling may be stabilized by providing intermittent doses of the compound of Formula (I).

The compound of Formula (I) is thought to selectively bind the GABA_{A} receptor at a site that is distinct from those of barbiturates and benzodiazepines. Moreover, unlike benzodiazepines, which bind exclusively at synaptic GABA_{A} receptors to mediate short bursts of ‘phasic’ inhibitor, the compound of Formula (I) is thought to bind at extra synaptic GABA_{A} receptors to mediate ‘tonic’ inhibition, which regulates network-level excitability and acts on a longer timescale. Modulating tonic inhibition is relevant to multiple disease states, including seizure and mood disorders.

The side-effects of effective doses of the compound of Formula (I) are believed to be limited. The abuse potential of effective doses of the compound of Formula (I) are believed to be limited.

### Definitions

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

As used herein, a “pure isomeric” compound or “isomerically pure” compound is substantially free of other isomers of the compound. The term “pure isomeric” compound or “isomerically pure” denotes that the compound comprises at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the compound with the specified structure. In certain embodiments, the weights are based upon total weight of all isomers of the compound.

As used herein, a “pure stereoisomeric” compound or “stereoisomerically pure” compound is substantially free of other stereoisomers of the compound. Thus, the composition is substantially free of isomers that differ at any chiral center. If the compound has multiple chiral centers, a substantial majority of the composition contains compounds having identical stereochemistry at all of the chiral centers. The term “pure stereoisomeric” compound or “stereoisomerically pure” denotes that the compound comprises at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the compound with the specified stereochemistry. In certain embodiments, the weights are based upon total weight of all stereoisomers of the compound.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the enantiomer. In certain embodiments, the weights are based upon total weight of all enantiomers or stereoisomers of the compound.

Compounds described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; may be any isotopic form, including ¹⁴N and ¹⁵N; O may be in any isotopic form, including ¹⁶O and ¹⁸O; and the like.

As used herein, the terms “modulation” and “potentiation” of GABA_{A} receptor function refer to the inhibition or stimulation of GABA_{A} receptor function. A “modulator” or “potentiator” may be, for example, an agonist, partial agonist, antagonist, or partial antagonist of the GABA receptor. The “modulator” or “potentiator” may act at the active site or at an allosteric site on a GABA receptor. It may promote or inhibit ligand binding. It may facilitate or attenuate ligand-mediated, e.g., GAB A-mediated, signaling.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, el al., J. Pharm. Sci. (1977) 66(1): 1-79.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be 2H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e., 3H, and carbon-14, i.e., ¹⁴C, are particularly useful for this purpose in view of their ease of incorporation and ready means of detection. Further, compounds may be prepared that are substituted with positron emitting isotopes, such as ¹¹C, ¹⁸F, ¹⁵O, and ¹³N, and would be useful in Positron Emission Topography (PET) studies for examining substrate receptor occupancy. All isotopic variants of the compounds provided herein, radioactive or not, are intended to be encompassed within the scope of the invention.

“Stereoisomers”: It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers”, and those that are non-superimposable mirror images of each other are termed “enantiomers.” When a compound has an asymmetric center, for example, and an atom, such as a carbon atom, is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of n electrons and an atom (usually H). For example, enols and ketones are tautomers because they are rapidly interconverted by treatment with either acid or base. Another example of tautomerism includes the aci- and nitro-forms of phenylnitromethane that are likewise formed by treatment with acid or base. Tautomeric forms may be relevant to the attainment of the optimal chemical reactivity and biological activity of a compound of interest.

A “subject” to which administration is contemplated includes, but is not limited to, a human (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

In general, the “effective amount” of a compound refers to an amount sufficient to elicit the desired biological response, e.g., to treat a CNS-related disorder, is sufficient to induce anesthesia or sedation. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, “providing” to a subject a compound or a composition containing a compound includes both providing (1) the compound itself, or a composition containing the compound itself, and (2) providing a prodrug, analog, or derivative of the compound, or a composition containing the prodrug, analog, or derivative of the compound, in which the prodrug, analog, or derivative is converted in to the compound in vivo.

As used herein, “maximum tolerated dose” means the highest dose of a compound or composition that does not cause unacceptable side effects.

### Compositions Containing the Compound of Formula (I)

The methods of the invention include providing to a subject a composition, e.g., a pharmaceutic composition, that contains a therapeutically effective amount of a compound of Formula (I):

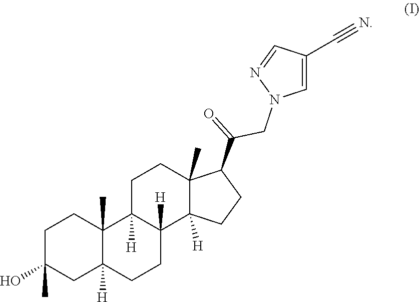

The composition may contain an isomerically pure form of a compound of Formula (I). The composition may be chemically pure, i.e., free from other molecules or chemical species. For example, the other molecule or chemical species may have a distinct chemical formula, structural formula, empirical formula, molecular formula, or condensed formula. The composition may have a defined level of chemical purity. For example, the compound of Formula (I) may be present at at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the total amount of a mixture that includes the compound of Formula (I) and one or more distinct molecules or chemical species.

The composition may contain the compound of Formula (I) at a defined level of isomeric purity, i.e., the composition may contain the compound of Formula (I) at a level in relation to an isomeric form of the compound. For example, the compound of Formula (I) may be present at at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the total amount of isomeric molecules that include the compound of Formula (I) and an isomer thereof.

The composition may be isomerically pure with respect to all isomers. The composition may be isomerically pure with respect to one or more particular types of isomers. The composition may be substantially free of structural isomers or a particular type of structural isomers, such as a regioisomers. The composition may be substantially free of stereoisomers or a particular type of stereoisomers, such as enantiomers or diastereomers.

The composition may contain the compound of Formula (I) at a level of isomeric purity to achieve preferential modulation of one of more subtypes of GABA_{A} receptors as compared to one or more different subtypes of GABA_{A} receptors. For example, the composition may contain the compound of Formula (I) at a level of isomeric purity to achieve preferential modulation of an α4β3δ GABA_{A} receptor as compared to an α1β2γ2 GABA_{A} receptor. The compound of Formula (I) may be present at at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the total amount of isomeric molecules that include the compound of Formula (I) and an isomer thereof.

The composition may contain the compound of Formula (I) and be substantially free of stereoisomers. The stereoisomer may differ from Formula (I) at one, two, three, four, five, six, seven, or eight chiral centers. The stereoisomer may be a diastereomer or an enantiomer. For example, the stereoisomer may be a compound of Formulas (II) or (III):

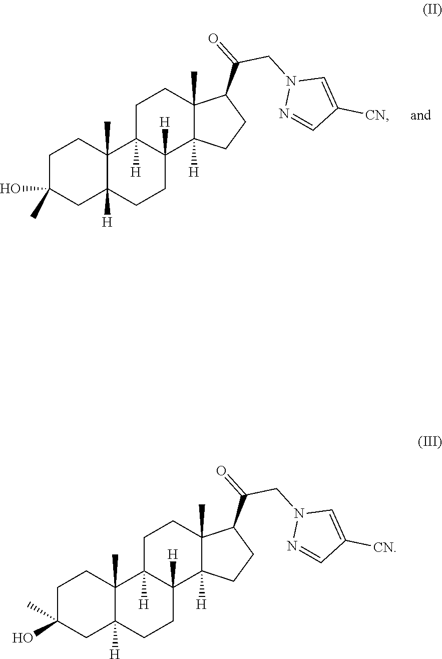

The composition may contain one or more stereoisomers of the compound of Formula (I), such as a compound of Formula (II) or (III), at less than 5%, less than 4%, less than 3%, less than 2%, less than 1%, less than 0.5%, or less than 0.1% of the total of the compound of Formula (I) and the one or more stereoisomers thereof. The composition may contain the compound of Formula (I) and one or more stereoisomer thereof at a ratio of at least 19:1, 20:1, 25:1, 30:1, 40:1, 50:1, 100:1, 200:1, 500:1, or 1000:1.

The pharmaceutical composition containing the compound of Formula (I) may be in a form suitable for oral use, for example, as tablets, troches, lozenges, fast-melts, aqueous or oily suspensions, dispersible powders or granules, emulsions, hard or soft capsules, syrups or elixirs. Compositions intended for oral use may be prepared according to any method known in the art for the manufacture of pharmaceutical compositions and such compositions may contain one or more agents selected from sweetening agents, flavoring agents, coloring agents and preserving agents, in order to provide pharmaceutically elegant and palatable preparations. Tablets contain the compounds in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration in the stomach and absorption lower down in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108; 4,166,452; and 4,265,874, the contents of which are incorporated herein by reference, to form osmotic therapeutic tablets for control release. Preparation and administration of compounds is discussed in U.S. Pat. No. 6,214,841 and U.S. Pub. No. 2003/0232877, the contents of which are incorporated herein by reference.

Formulations for oral use may also be presented as hard gelatin capsules in which the compounds are mixed with an inert solid diluent, for example calcium carbonate, calcium phosphate or kaolin, or as soft gelatin capsules in which the compounds are mixed with water or an oil medium, for example peanut oil, liquid paraffin or olive oil.

An alternative oral formulation, where control of gastrointestinal tract hydrolysis of the compound is sought, can be achieved using a controlled-release formulation, where a compound of the invention is encapsulated in an enteric coating.

Aqueous suspensions may contain the compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compounds in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compounds in admixture with a dispersing or wetting agent, suspending agent and one or more preservatives. Suitable dispersing or wetting agents and suspending agents are exemplified, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

In certain embodiments, the formulation is not a sustained release formulation. In certain embodiments, the formulation is not injectable. In certain embodiments, the formulation does not contain particles having a D50 (volume weighted median diameter) of less than 10 microns. In certain embodiments, the formulation does not contain a polymer surface stabilizer. In certain embodiments, the formulation is not an aqueous suspension.

The composition may be formulated a single daily dosage. The composition may be formulated for multiple daily dosages, e.g., two, three, four, five, six or more daily dosages.

The composition may be provided to the subject according to any dosing schedule. The composition may be provided once per day. The composition may be provided multiple times per day. The composition may be provided two times, three times, four times, five times, six times, or more per day.

### Providing a Composition Containing the Compound of Formula (I) to a Subject

Methods of the invention may include providing a composition containing a therapeutically effective amount of the compound of Formula (I) to a subject. The composition may include the compound of Formula (I) itself. Alternatively or additionally, the composition may include a prodrug, analog, or derivative of the compound of Formula (I) that is converted to the compound of Formula (I) in the body of the subject.

The composition may be provided according to a dosing regimen. A dosing regimen may include one or more of a dosage, a dosing frequency, and a duration. Preferably, the compound of Formula (I) is provided at a dose of about 5 to about 90 mg. The compound of Formula (I) may be provided at a dose between about 5 mg and about 1000 mg. For example, the compound of Formula (I) may be provided at a dose of 5 mg, 15 mg, 30 mg, 60 mg, 90 mg, 100 mg, 125 mg, 150 mg, 175 mg, 200 mg, 250 mg, 300 mg, 350 mg, 400 mg, 500 mg, 600 mg, 700 mg, 800 mg, or 1000 mg. The compound of Formula (I) may be provided in the composition at the maximum tolerated dose.

Doses may be provided at any suitable interval. For example and without limitation, doses may be provided once per day, twice per day, three times per day, four times per day, five times per day, six times per day, eight times per day, once every 48 hours, once every 36 hours, once every 24 hours, once every 12 hours, once every 8 hours, once every 6 hours, once every 4 hours, once every 3 hours, once every two days, once every three days, once every four days, once every five days, once every week, twice per week, three times per week, four times per week, or five times per week.

The dose may be provided in a single dosage, i.e., the dose may be provided as a single tablet, capsule, pill, etc. Alternatively, the dose may be provided in a divided dosage, i.e., the dose may be provided as multiple tablets, capsules, pills, etc.

The dosing may continue for a defined period. For example and without limitation, doses may be provided for at least 1 week, at least 2 weeks, at least 3 weeks, at least 4 weeks, at least 6 weeks, at least 8 weeks, at least 10 weeks, at least 12 weeks, at least 4 months, at least 5 months, at least 6 months, at least 8 months, at least 10 months, at least 12 months or more.

### Preferential Modulation of GABA_{A} Receptor Subtypes

As indicated above, methods of the invention may include providing a composition containing a compound of Formula (I) at a therapeutically effective amount to preferentially modulate one or more GABA_{A} receptor subtypes over other GABA_{A} receptor subtypes. GABA_{A} receptors are ligand-gated ion channels that selectively allow Cl-ions to pass through the plasma membrane upon binding of GABA. GABA_{A} receptors are expressed in neurons throughout the central nervous system (CNS), Leydig cells, placenta, immune cells, liver, bone growth plates, and other endocrine tissues.

Structurally, GABA_{A} receptors are pentamers that include five polypeptide subunits. The polypeptide subunits are encoded by 19 genes that are grouped as follows based on sequence similarity: α(1-6), β(1-3), γ(1-3), δ, ε, θ, π, and ρ(1-3). Most subtypes are heteropentamers that include two copies of one type of a subunit, two copies of one type of β subunit, and one copy of one type of γ, δ, ε, θ, or π subunit; other subtypes are homopentamers or heteropentamers of p subunits. Known subtypes of GABA_{A} receptors include α1β1γ2, α1β2γ2, α1β3γ2, α2β1γ2, α2β2γ2, α2β3γ2, α3β1γ2, α3β2γ2, α3β3γ2, α4β1γ2, α4β3δ, α4β3γ2, α5β1γ2, α5β2γ2, α5β3γ2, α6β1γ2, α6β2γ2, and α6β3γ2. GABA_{A} receptor subtypes vary among tissue types and anatomical regions of the CNS, and subtypes may be associated with specific functions. In addition, GABA_{A} receptor subtypes may vary between normal and malignant cells of the same tissue type.

The active site of a GABA_{A} receptor is the binding site for GABA and for drugs such as muscimol, gaboxadol, and bicuculline. GABA_{A} receptors also have several allosteric binding sites that are the targets of other drugs, including benzodiazepines, nonbenzodiazepines, neuroactive steroids, barbiturates, ethanol, inhaled anaesthetics, and picrotoxin. Thus, the activity of GABA_{A} receptors is controlled by binding of molecules to both the active and allosteric binding sites. The structure, function, and regulation of GABA_{A} receptors are known in the art and described in, for example, Sigel E., and Steinmann, M. E., Structure, Function, and Modulation of GABA_{A} Receptors, J. Biol. Chem. 287:48 pp. 40224-402311 (2012), doi: 10.1074/jbc.R112.386664, the contents of which are incorporated herein by reference.

The compositions of the invention may preferentially potentiate the activity of one or more GABA_{A} receptor subtypes, such as those described above, relative to other GABA_{A} receptor subtypes. In certain embodiments, the compositions preferentially potentiate the activity of α4β3δ receptors compared to α1β2γ2 receptors. For example, as shown herein, isomerically pure compositions of CV-10155 preferentially modulate GABA_{A} receptors of the α4β3δ subtype compared to receptors of the α1β2γ2 subtype

The compositions of the invention may potentiate one or more GABA_{A} receptor subtypes by any mechanism. For example, and without limitation, the isomerically pure form a compound may potentiate a GABA_{A} receptor by allosteric modulation, activation, or inhibition. The allosteric modulation may be positive or negative.

The preferential activity of a composition on one or more GABA_{A} receptor subtypes as compared to other GABA_{A} receptor subtypes may be measured by any suitable means. Activity may be measure using in vitro assays or in vivo assays. For example and without limitation, methods of measuring the effect of modulators on GABA_{A} receptor activity include anticonvulsant assays, binding assays, fluorescence membrane potential assays, immune response assays, intracranial self-stimulation assays patch clamps assays, proliferation assays receptor occupancy assays seizure induction assays, e.g., using pentylenetetrazol (PTZ) or maximal electroshock (MES), and survival assays. Such assays are known in the art and described in, for example, International Publication No. WO 2016/061527; Ghisdal P., et al., Determining the relative efficacy of positive allosteric modulators of the GABA_{A} receptor: design of a screening approach, J Biomol Screen. 2014 March; 19(3):462-7. doi: 10.1177/1087057113501555, Epub 2013 August 29; Tian J., et al., Clinically applicable GABA receptor positive allosteric modulators promote B-cell replication, Sci Rep. 2017 Mar. 23; 7(1):374. doi: 10.10381s41598-017-00515-y; and Tian J., et al., A Clinically Applicable Positive Allosteric Modulator of GABA Receptors Promotes Human 13-Cell Replication and Survival as well as GABA's Ability to Inhibit Inflammatory T Cells, J Diabetes Res. 2019 Feb. 26; 2019:5783545, doi: 10.1155/2019/5783545, the contents of each of which are incorporated herein by reference.

The preferential activity of a composition on one or more GABA_{A} receptor subtypes as compared to other GABA_{A} receptor subtypes may be expressed by any suitable means. For example and without limitation, the preferential activity may be indicated by a comparison of EC50 values or binding affinity values.

In certain embodiments, compositions of the invention have an EC50 for α4β3δ GABA_{A} receptors that is lower than the EC50 for α1β2γ2 GABA_{A} receptors. The EC50 for α4β3δ GABA_{A} receptors may be lower than the EC50 for α1β2γ2 GABA_{A} receptors by about 2-fold, about 3-fold, about 4-fold, about 5-fold, about 6-fold, about 7-fold, about 8-fold, about 10-fold, about 20-fold, about 50-fold, about 100-fold, about 200-fold, about 500-fold, or about 1000-fold.

In certain embodiments, compositions of the invention have an EC50 for α4β3δ GABA_{A} receptors that is less than about 50%, less than about 40%, less than about 30%, less than about 25%, less than about 20%, less than about 15%, less than about 10%, less than about 5%, less than about 4%, less than about 3%, less than about 2%, less than about 1%, less than about 0.5%, less than about 0.2%, or less than about 0.1% of the EC50 for α1β2γ2 GABA_{A} receptors.

In certain embodiments, compositions of the invention have a binding affinity (which may be expressed, e.g., as a dissociation constant KD) for α4β3δ GABA_{A} receptors that is lower than the binding affinity for α1β2γ2 GABA_{A} receptors. The binding affinity for α4β3δ GAB AA receptors may be lower than the binding affinity for α1β2γ2 GABA_{A} receptors by about 2-fold, about 3-fold, about 4-fold, about 5-fold, about 6-fold, about 7-fold, about 8-fold, about 10-fold, about 20-fold, about 50-fold, about 100-fold, about 200-fold, about 500-fold, or about 1000-fold.

In certain embodiments, compositions of the invention have an binding affinity for α4β3δ GABA_{A} receptors that is less than about 50%, less than about 40%, less than about 30%, less than about 25%, less than about 20%, less than about 15%, less than about 10%, less than about 5%, less than about 4%, less than about 3%, less than about 2%, less than about 1%, less than about 0.5%, less than about 0.2%, or less than about 0.1% of the binding affinity for α1β2γ2 GABA_{A} receptors.

In certain embodiments, compositions of the invention have an EC50 for α4β3δ GABA_{A} receptors that is below a defined value. For example and without limitation, the composition may have an EC50 for α4β3δ GABA_{A} receptors that is less than about 1 less than about 500 nM, less than about 400 nM, less than about 300 nM, less than about 200 nM, less than about 100 nM, less than about 50 nM, less than about 25 nM, less than about 10 nM, less than about 5 nM, less than about 2.5 nM, less than about 1 nM, less than about 0.5 nM, less than about 0.25 nM, or less than about 0.1 nM.

In certain embodiments, compositions of the invention have a binding affinity for α4 β3δ GABA_{A} receptors below a defined value. For example and without limitation, the composition may have an binding affinity for α4β3δ GABA_{A} receptors that is less than about 1 μM, less than about 500 nM, less than about 400 nM, less than about 300 nM, less than about 200 nM, less than about 100 nM, less than about 50 nM, less than about 25 nM, less than about 10 nM, less than about 5 nM, less than about 2.5 nM, less than about 1 nM, less than about 0.5 nM, less than about 0.25 nM, or less than about 0.1 nM.

### EXAMPLES

### Example 1—a Phase 1 Study of the Safety, Tolerability and Pharmaceutics of ATN-10155 in Health Volunteers

Study Description: Rationale, and Background

A Phase I study is conducted of the compound ATN-10155, having the structure of Formula (I), as a positive allosteric modulator of the GABA_{A} receptor in humans.

The compound of Formula (I) is believed to be inactive at the classical nuclear progesterone receptor. The compound of Formula (I) is believed to have binding affinity at the GABA-gated Cl-channel, the kappa opioid receptor, the 5-HT_{2A} receptor, and the androgen receptor. The compound of Formula (I) has previously shown activity at all 18 GABA_{A} receptor channels and the kappa opioid receptor. The compound of Formula (I) had previously shown no activity at the 5-HT_{2A} receptor. Activity at the androgen receptor is has not yet been well studied.

The compound of Formula (I) is also believed to show antiepileptic activity in maximal electroshock and pentylenetetrazol seizure models, antidepressant properties in the forced swim test, and anxiolytic properties in the elevated plus maze test. The compound of Formula (I) has been shown to be well tolerated in rodent models at efficacious doses.

The study is a first in-human study of ATN-10155 is designed to assess the safety, tolerability, and pharmacokinetics of ATN-10155 following administration of two phases: a single ascending dose phase and multiple ascending dose phase. The study is a randomized, double-blind study with participants in each cohort receiving active drug or matching placebo. The study evaluates the safety, tolerability, and pharmacokinetics of ATN-10155 following the single and multiple dose phases in healthy volunteers. Administering ascending single and multiple ascending doses in healthy volunteers is a standard and appropriate approach to assessing the safety, tolerability, and pharmacokinetics of a new chemical entity.

Single Ascending Dose—the Single Ascending Dose portion comprises five or more cohorts with eight participants in each cohort, 6 actively receiving ATN-10155 and 2 receiving a placebo. A single morning dose is administered. Blood samples are taken prior to and through 48 hours following dosing. The drug is administered as 5 mg, 15 mg, 30 mg, 60 mg, and 90 mg doses. Additional cohorts are added if the maximum tolerated dose is not reached and at a level between the non-tolerated dose and the previously tolerated dose. Intermediate cohorts are added if deemed necessary by a Safety Review Committee. Sentinel dosing is employed for each cohort (1 active, 1 placebo). Sentinel dosing is not employed once the maximum tolerated dose is determined or if an intermediate cohort is added that that is lower than the dose of the previous cohort.

Blood samples are taken prior to and through 48 hours following dosing. All cohorts are dosed after at least a 10-hour fast. Cohort 3 is dosed twice, fasted and fed (dosing separated by approximately one week) to assess the effect of food on absorption. Dosing is separated by approximately one week to allow for an assessment of safety and tolerability.

Once the maximum tolerated dose is determined, participants are asked to return for a second dose administration in the evening (dosing separated by approximately one week) to assess diurnal effects on pharmacokinetics. All returning participants are administered active treatment. If a significant food effect is seen with Cohort 3, some or all participants in the maximum tolerated dose cohort are asked to return a third time to further define the food effect at the maximum tolerated dose.

Multiple Ascending Dose—the Multiple Ascending Dose phase involves up to three cohorts with twelve participants in each cohort, 9 actively receiving ATN-10155 and 3 receiving a placebo. Participants are given a single daily dose for 7 days. Blood samples are taken prior to each dose, trough 24 hours following the first dose, and through 48 hours the last dose. Dosing is separated by approximately 2 weeks to allow for an assessment of safety and tolerability.

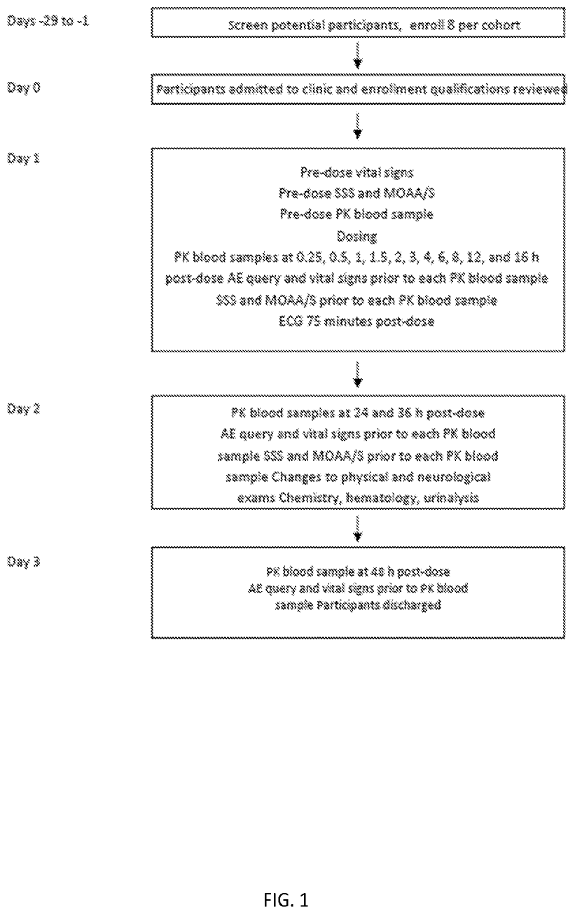

FIG. 1 is a flow chart of a single ascending dose study design of the compound of Formula (I).

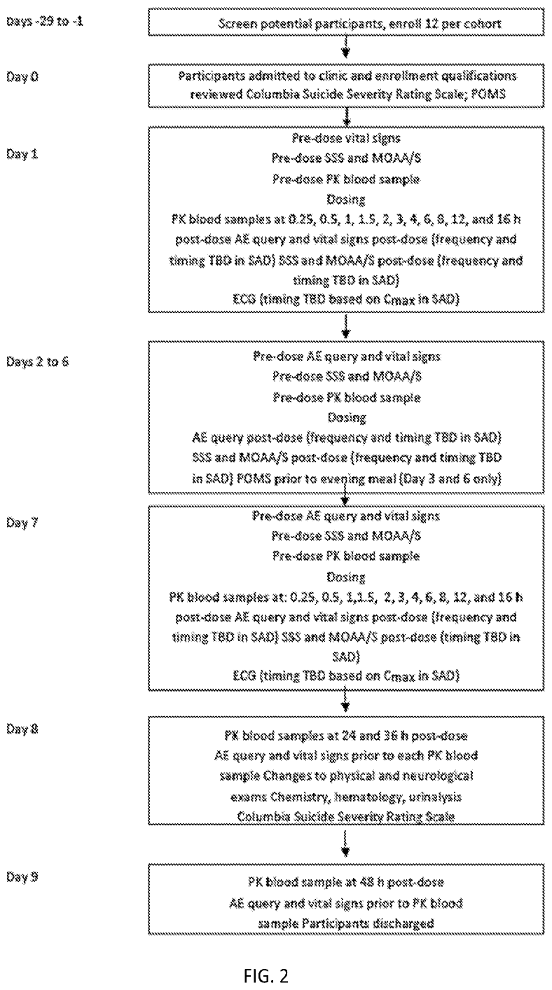

FIG. 2 is a flow chart of a multiple ascending dose study design of the compound of Formula (I).

Risk/Benefit Assessment and Justification for Dose

Known potential Risks—Prior to this Phase I study, in a comprehensive toxicology program involving repeat-dose toxicity for up to four weeks, the primary effects observed after administration of ATN-10155 were consistent with supra-pharmacological effects on the GABA_{A} receptor, e.g. ataxia and prostration at the mid and high dose levels. The no-observed-adverse-effect-level doses were 7.5/mg/kg/day and 6 mg/kg/day in rats and dogs, respectively. Results in in vivo safety pharmacology studies show that ATN-10155 does not affect cardiovascular or respiratory function. Two in vitro genetic toxicology studies showed no positive findings. Based on these data, ATN-10155 is considered to have a low risk of causing significant adverse events in humans.

Known potential benefits—Prior to this Phase I study, there were no known benefits for participant s in this study, other than the benefit of medical evaluation throughout the study. Participation in this study may help generate future benefits for patients experiencing CNS disorders for which the compound is studied and approved.

Assessment of Risks and Benefits—The safety and tolerability data demonstrated in non-clinical toxicology studies, the low starting dose, the use of sentinels, and the conservative dosing increments all contribute to risk mitigation and to the safe conduct of this Phase I study.

Justification for Dose—Based on the nonclinical toxicology data, there is not an unreasonable risk of adverse effects. Using the FDA guidance “Estimating the Maximum Safe Starting Dose in Initial Clinical Trials for Therapeutics in Adult Healthy Volunteers” (July, 2005), the calculated human equivalent dose would be 1/2 mg/kg/day or 3/24 mg/kg/day, respectively, based on the 28-day rat no-observed-adverse-effect-level dose of 7.5 mg/kg/day or the 28-day dog no-observed-adverse-effect-level dose of 6 mg/kg/day, respectively. For both species the no-observed-adverse-effect-level dose was the highest dose tested. For a 60 kg participant, this would result in a maximum safe starting dose of (human equivalent dose/10) of 7.2 mg/day or 19/6 mg/day. The starting doses in this Phase 1 study are 5 mg, equivalent to 0.83 mg/kg/day for a 60 kg participant. Based on allometric scaling using pharmacokinetic data in dogs, prior to this Phase I study, the anticipated plasma exposure following a 5 mg dose in humans is a Cₘₐₓ of ˜15 ng/ml and an AUC of ˜300 h*ng/mL. These anticipated Cₘₐₓ and AUC values are ˜80- and 50-fold lower, respectively, than that observed at the no-observed-adverse-effect-level dose in dogs.

Objectives

The primary objective is to determine the safety and tolerability of orally administered single and multiple doses of ATN-10155.

The secondary objective is to determine the pharmacokinetic profile of ATN-10155 following single and multiple doses.

Study Endpoints, Assessment and Procedures—The following endpoints are monitored:

Safety and tolerability—A physical and neurological exam is done as part of the eligibility evaluation at screening. Any changes in physical exams is documented upon admission and prior to discharge (which may be done on Day 2 or Day 3). Safety and tolerability is assessed through observed and reported adverse events, vital signs, ECG, blood chemistry, hematology, urinalysis, the Stanford Sleepiness Scale, the Modified Observer's Alertness/Sedation Scale, and in the multiple ascending dose portion, the short form Profile of Mood States and the Columbia-Suicide Severity Scale, as detailed below.
Observed and reported adverse events.
Vital signs: blood pressure pulse, pulse oximetry. Systolic blood pressure, diastolic blood pressure, pulse, and peripheral arterial oxygen saturation (pulse oximetry) is recorded prior to every PK blood sample (including pre-dose) in the single ascending dose portion. Participants rest for approximately 5 minutes prior to assessments. Vital signs may be taken in either the seated or supine position but always in the same position for any given participant. In the multiple ascending dose portion, the post-dose vital signs are taken daily prior to dosing and 4 times following dosing (times based on single ascending dose results).
Clinical laboratory test: laboratory tests are performed at screening, on admission (Day 0), and prior to discharge. If female, a serum pregnancy test is done at screening and a serum or urine pregnancy test on admission (Day 0). If a post-menopausal female, follicle stimulating hormone levels is measured at screening. At screening, tests for Human Immunodeficiency Virus, Hepatitis B Virus, and Hepatitis C Virus are conducted. At screening and admission, urine drug and alcohol screen, and cotinine screen are done.
   Hematology: HCT, Hgb, MCH, MCHC, MCV, PLT, RBC, WBC, and differentials.
   Biochemistry: Albumin, alkaline phosphatase, ALT, AST, chloride, creatinine (enzymatic), glucose random, LDH, potassium, sodium, total bilirubin, urea (BUN), uric area.
   Urinalysis: Chemical and microscopic analysis.
ECG—twelve-lead ECGS are taken at screening, on Day 1 prior to dosing and at the anticipated tₘₐₓ, and in the multiple ascending dose portion on Day 7 prior to dosing and at the anticipated tₘₐₓ. ECGs should be taken after participants lie supine for approximately 5 minutes.
Sedation—Sedation is assessed using the self-reported Sanford Sleepiness Scale (SSS) as well as by the Modified Observer's Assessment of Alertness/Sedation scale (MOAA/S). Sedation is assessed on the same schedule as vital signs: prior to every PK blood sample (including pre-dose) in the single ascending dose portion. The MOAA/S is conducted first followed by the SSS and then AE questioning, vitals, and PK sampling. In the multiple ascending dose portion, sedation (and other mood factors) is also assessed using the Profile of Mood States (POMS). The POMS is completed upon admission, and on Days 3 and 6. The POMS is completed by the participants immediately before the evening meal. In the cohort dosed at multiple ascending doses in the evening, the POMS will not be administered.
Columbia-Suicide Severity Rating Scale (multiple ascending dose portion)

Pharmacokinetics—In both the single ascending dose and multiple ascending dose portions, pharmacokinetic parameters include the maximum observed concentration (Cₘₐₓ), the time of the maximum concentration (tₘₐₓ), half-life, t_{1/2}, and the area under the plasma concentration curve from 0 hours to 28 hours (AUC₀₋₄₈). In the single ascending dose portion, the effect of food is determined in cohort 3, which is dosed under both fed and fasted conditions. In the multiple ascending dose portion, AUC₀₋₂₄ is calculated on days 1 and 7. The accumulation ratio (Rac) is calculation from both Day 7 AUC₀₋₂₄/Day 1 AUC_{0-24 (}[R_{act(AUC)}]) and Day 7 Cₘₐₓ/Day 1 Cₘₐₓ [R_{act}(Cₘₐₓ)].
Cₘₐₓ, tₘₐₓ, t_{1/2}, and AUC₀₋₄₈
AUC₀₋₂₄ (multiple ascending dose days 1 to 7)
Effect of food (single ascending cohort 3)
In the multiple ascending dose: accumulation ratio based on both Cₘₐₓ [R_{act(Cmax)}] and AUC₀₋₂₄ [R_{act(AUC)}]

Blood Sampling Times—blood is sampled according to the following schedules.

Single Ascending Dose schedule:
Day 1: prior to dosing and following dosing at 0.25, 0.5, 1, 1.5, 2, 3, 4, 6, 8, 12, 16 hours
Day 2: 24 and 36 hours following dosing; and
Day 3: 48 hours following dosing

Multiple Ascending Dose schedule:
Day 1: prior to dosing and following dosing at 0.25, 0.5, 1, 1.5, 2, 3, 4, 6, 8, 12, 16 hours
Day 2-6: prior to dosing
Day 7: prior to dosing and following dosing at 0.5, 1, 2, 4, 6, 8, 12, and 16 hours
Day 8: 24 and 36 hours following the last dose; and
Day 9: 48 hours following the last dose

The sample times and number of samples in later single ascending dose cohorts and multiple ascending dose cohorts may be altered based on earlier findings. All plasma samples are analyzed using a validated LC-MS/MS method at a GLP-certified bioanalytical laboratory.

Pharmacokinetic parameters are only calculated for participants with not more than two missing analytical results over the course of the planned post-dose sampling period. Only participants with PK parameters under both fed and fasted conditions are included in the analysis of the effect of food. Only participants with PK parameters from both multiple ascending dose Day 1 and multiple ascending dose Day 7 are included in the analysis of the accumulation factor. All participants who received at least one dose of the investigational product are included in the safety analysis.

Study Population and Criteria

Inclusion Criteria—to be eligible a participant meets the following criteria:
Healthy, male or female, 18 to 55 years of age, inclusive, at screening.
Weigh at least 50 kg and have a MBI≤33 g/m².
If female of childbearing potential and involved in any sexual intercourse that could lead to pregnancy, participant agrees that an effective contraceptive method will be used, from at least 4 weeks prior to Day 0 until at least 4 weeks or until first menses after the last investigational product administration. Effective contraceptive methods, include any one of the following: hormonal contraceptives (combined oral contraceptive, patch, vaginal ring, injectable, or implant), intrauterine devices or intrauterine systems, male partner(s) vasectomy, or double barrier method of contraception (male condom, female condom, cervical cap, diaphragm, or contraceptive sponge) in conjunction with spermicide. In the case of hormonal contraceptives, participants have been on a stable dose of hormonal contraceptives for at least 4 weeks before Day 0. A female of non-childbearing potential is defined as a female who is permanently sterile (e.g., tubal ligation or hysterectomy) or postmenopausal, which is defined as 12 consecutive months with no menses without an alternative medical cause and confirmed with FSH test at screening.
If female, have a negative serum pregnancy test at screening and negative serum or urine pregnancy test at Day 0.
Men who are able to impregnate a female agree to use medically acceptable methods of contraception during the study and for 4 weeks after the end of the study. Medically acceptable methods of contraception include using a condom with a female partner of child-bearing potential who is using oral contraceptives, hormonal patch, implant or injection, intrauterine device, or diaphragm with spermicide. Abstinence as a method of contraception is acceptable if it is in line with your preferred and usual lifestyle.
Medically healthy with no clinically significant medical history, vital signs, ECG, or physical examination findings or laboratory resultsr.
Is able and willing to participate and comply with all study procedures, including providing multiple blood samples.
Is capable of giving informed consent.

Exclusion Criteria—participants who meet the following criteria are excluded from the study:
Positive urine drug screen or alcohol results at Screening or Day 0
History or presence of clinically significant medical condition or disease.
Any history of major psychiatric disorders, including substance use disorders, according to the Diagnostic and Statistical Manual of Mental Disorders, 5th Edition (DSM-5) criteria.
Acute suicidality or history of suicidal behavior.
History of regular alcohol consumption within 12 months of the study defined as an average weekly intake of >21 alcoholic drinks/wee for men or >14 alcoholic drinks/week for women.
Use of tobacco-containing products over past 3 months of has a positive cotinine test at screening or on admission.
A positive Hepatitis B surface antigen or positive Hepatitis C antibody result at screening.
A positive test for human immunodeficiency virus (HIV) antibody at screening.
Alanine aminotransferase or aspartate aminotransferase levels greater than 1.5 times the upper limits of normal (ULN) at screening or Day 0. One retest is allowed.
Serum creatinine greater than 1.5 times the upper limit of normal (ULN) at Screening or Day 0. One retest is allowed.
Use of any prescription drug, therapy, except hormonal contraceptives, within the lesser of 2 weeks or five times the elimination half-life at admittance to the clinic or planned use during the duration of study participation.
An average (of three) corrected QT interval measurements corrected according to the Fredericia rule (QTcf>450 msec) during controlled rest at Screening of Day 1 (pre-dose), or family history of long QY syndrome.
Any clinically significant abnormalities in rhythm, conduction, or morphology of the resting ECG and any abnormalities in the 12-lead ECG that may interfere with the interpretation of QTc interval changes.
Use of non-prescription medication including vitamins and herbal supplements within the lesser of 72 hours or five times the elimination half-life at admittance to the clinic or planned use during the duration of study participation.
Consumption of caffeine/xanthine 12 hours before dosing and 2 hours after dosing on PK sampling days.
A female who is pregnant, breastfeeding, or plans to become pregnant during the study.
Donations of blood or significant loss of 50-499 mL with 30 days prior to first study drug administration or 500 mL within 56 days prior to first study drug administration.
Donation of plasma by plasmapheresis within 7 days prior to first study drug administration.
Plasma donation within 30 days prior to dosing of study drug.
Participation in another clinical trial with an investigational product within 28 days prior to admittance to the clinic.
Has a known or suspected allergy to ATN-10155 or any of the excipients.
Apart of oral contraceptives, no concomitant therapy is allowed, unless needed to treat an adverse event as determined.

Screen failures—screen failures are defined as individuals who consent to participate in the clinical trial but are not assigned to the investigational product because they did not meet all inclusion and exclusion criteria. Screening visit information is completed for these individuals as well as for individuals who consented and were eligible but were not randomized because the cohort was full, i.e. ‘backups’. Screen failures are rescreened once, if deemed acceptable.

Pregnancy reporting—If a participant, or the partner of a male participant, becomes pregnant during the study or within 4 weeks after completing the study, the participant should inform the study site as soon as possible. Upon confirmation of the pregnancy, a female participant is discontinued from the study. The pregnancy is reported within 24 hours of the confirmation of the pregnancy. Pregnancy is not itself an AE or an SAE, however, maternal/fetal complications or abnormalities are recorded as AEs of SAEs, as appropriate.

Treatment

Study Intervention—ATN-10155 is supplied as 5 and 30 mg tablets. The matching placebo tablets are prepared by compressing a direct blend of excipients. ATN-10155 an d placebo are packaged in HDPE bottles.

Dosing and Administration

In the single ascending dose portion of the study, participants are dosed in cohorts of six active and two placebo until the maximum tolerated dose is determined. The maximum tolerated dose administered is the highest dost administered that does not invoke stopping rules.

Based on animal no-observed-adverse-effect-level dose, the starting dose is 5 mg. The dose progression schedule is 5, 15, 30, 60, and 90 mg of ATN-10155. If the stopping rules are met by 90 mg, additional dose cohorts, always with sentinel dosing, are added pending a safety and tolerability review of the data. Once the stopping rules are met, an additional cohort is added at a dose between the non-tolerated dose and the previous dose; sentinel dosing is not required in this case.

All cohorts are dosed following at least a 10-hour fast and remain fasted for 2 hours following dosing. Cohort 3 is dosed a second time following ingestion of a standard high fat breakfast (no grapefruit or pineapple).

The dose administered in the multiple ascending dose portion is dependent upon the results from the single ascending dose portion. In the multiple ascending dose portion of the study, participants are administered daily morning doses of ATN-10155 from Day 1 to 7. Each dose is taken with or without food based on findings in the single ascending dose portion. The first cohort in the multiple ascending dose portion may be initiated prior to completion of the single ascending dose portion, but at a daily dose of no more than half of the last completed, tolerated, single ascending dose portion dose.

All doses are administered in the morning, with at least 240 mL of water.

Study Duration and End of Study

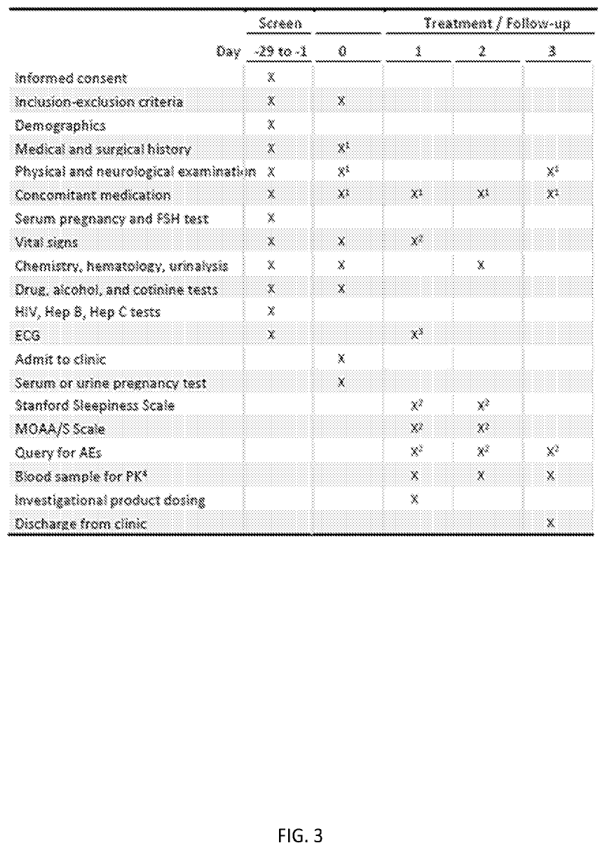

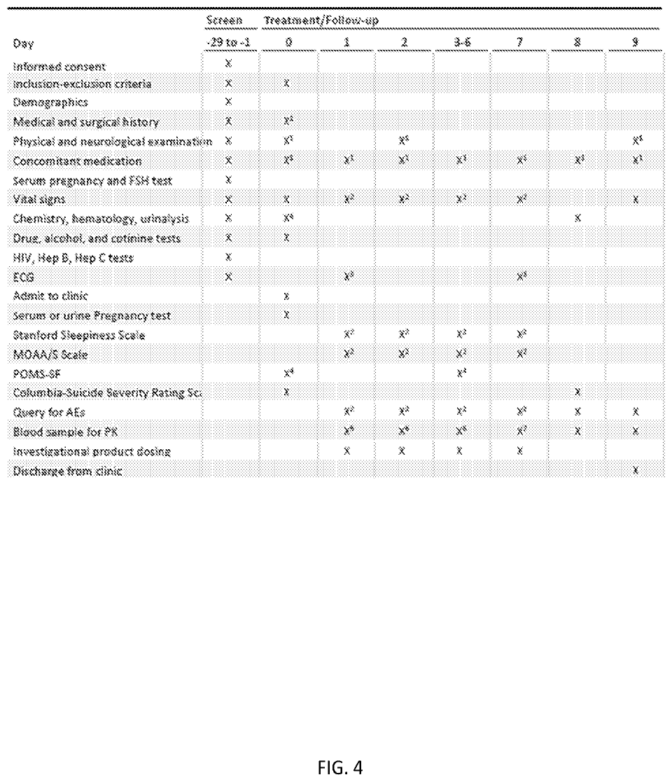

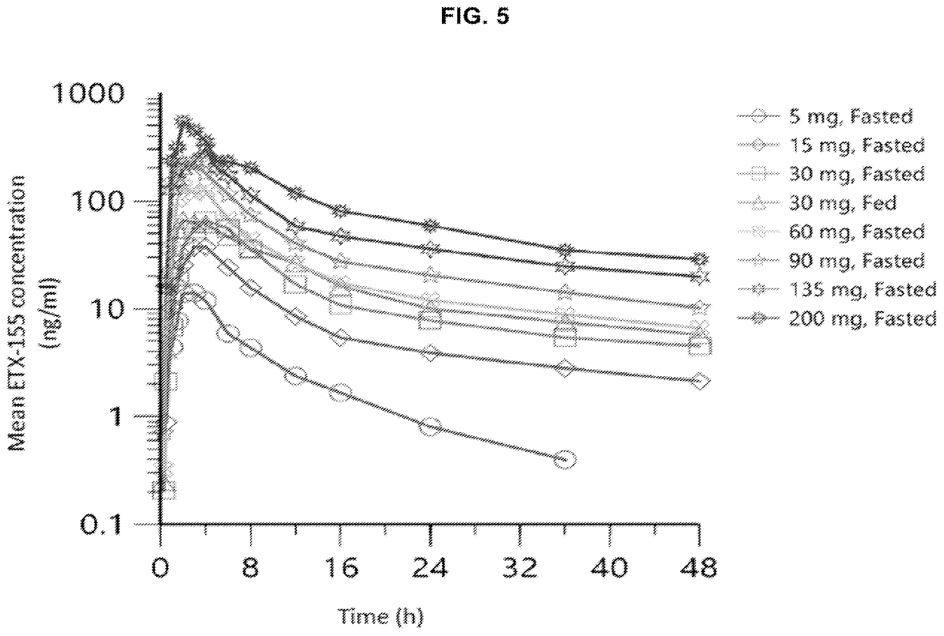

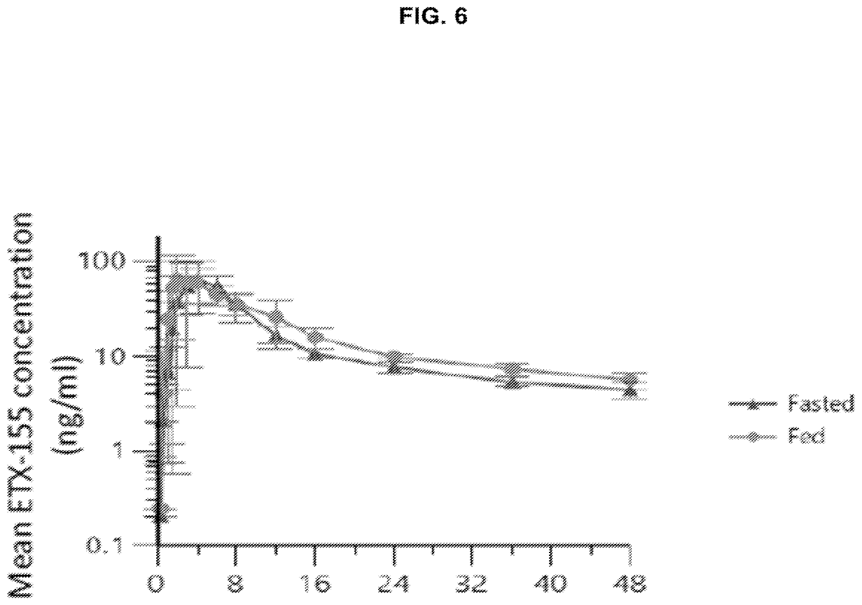

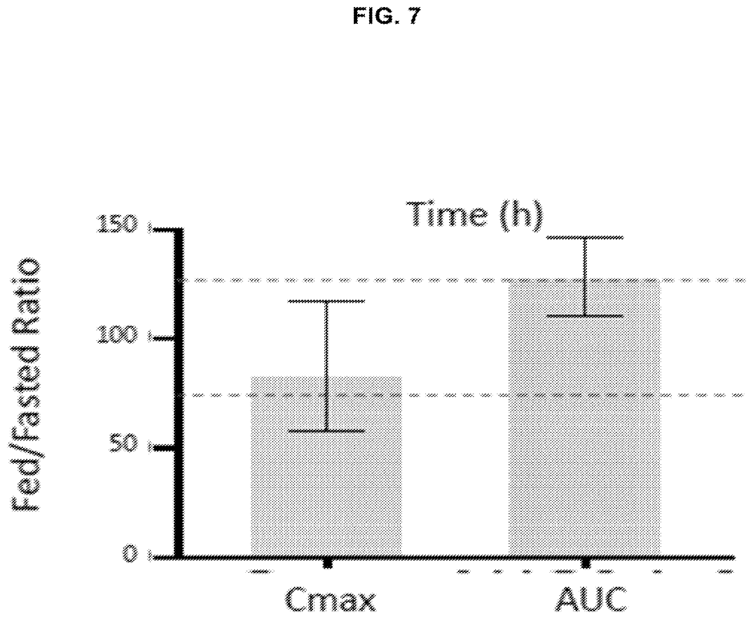

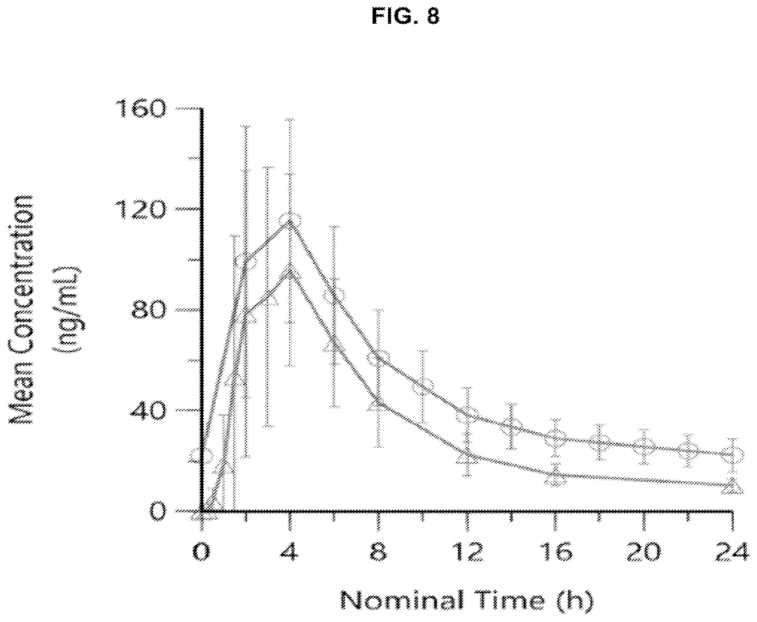

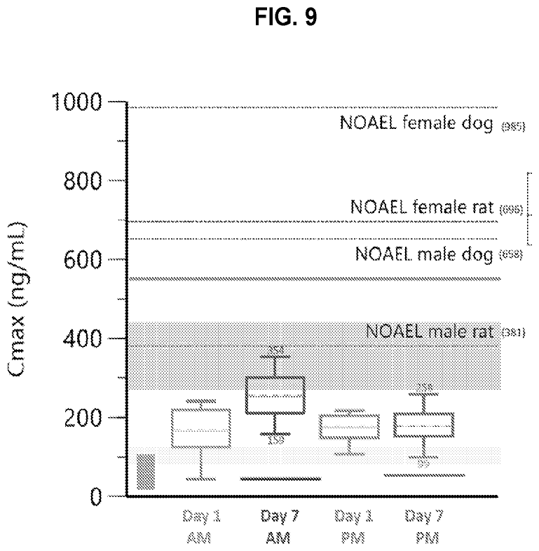

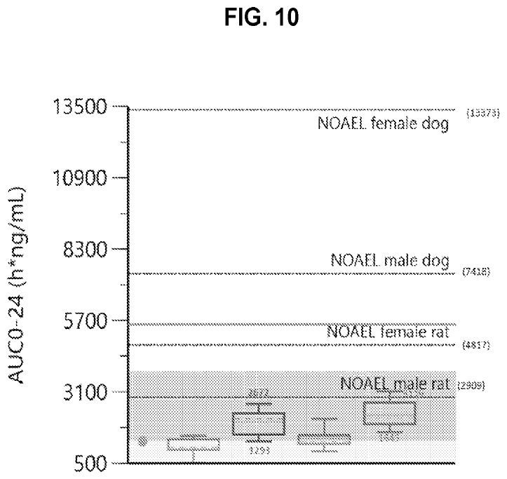

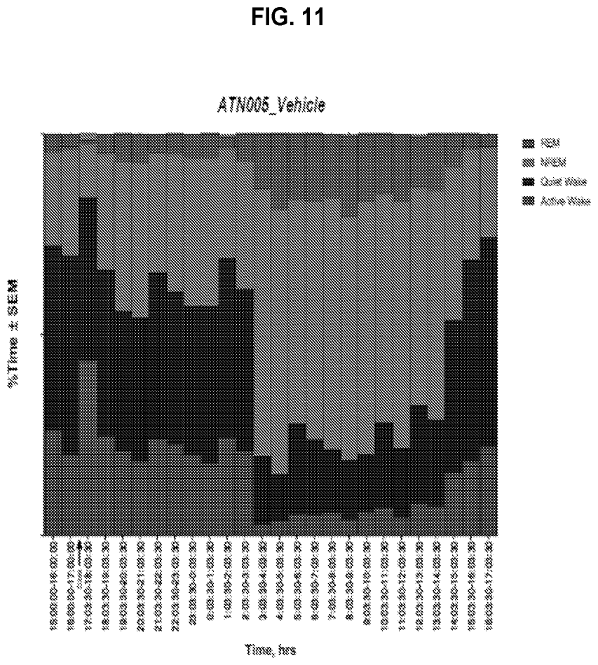

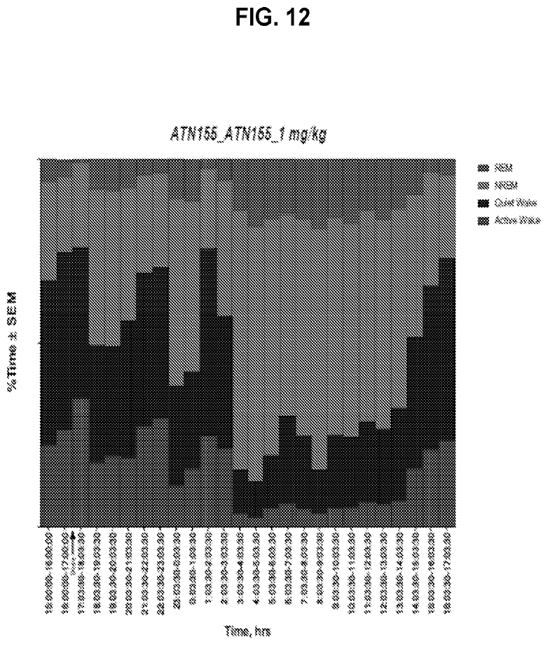

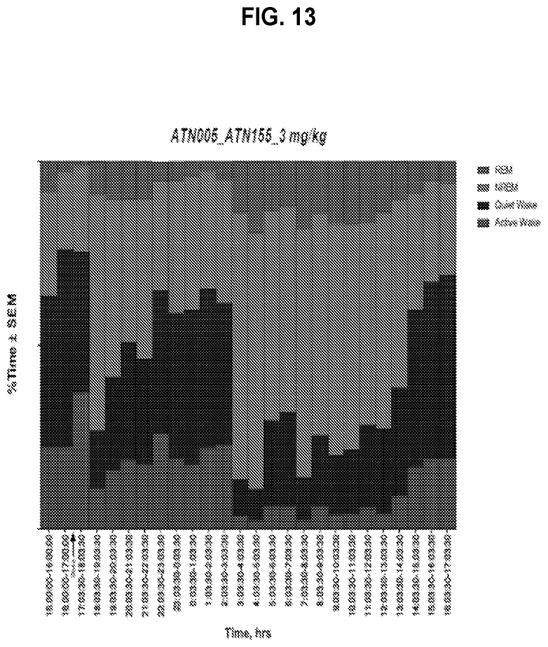

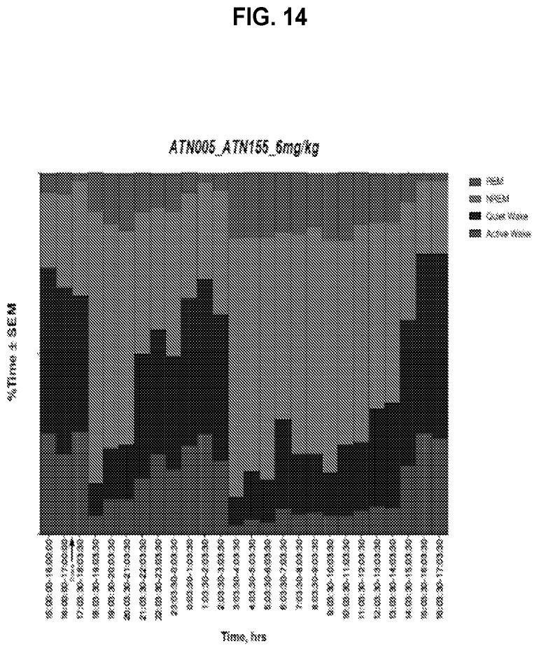

The study is conducted for approximately four months from first participant enrolled to last participant completed. A participant is considered to have completed the study if he or she has completed all phases of the study including the last visit or the last scheduled procedure shown in FIG. 1, which is “Table 1: SAD Schedule of Activities or FIG. 3, which is “Table 2: MAD schedule of activities.

Progressing—A safety review committee (SRC) evaluates the safety and tolerability of ATN-10155 after each cohort to determine progression to the next dose level, or choose a more conservative dose escalation rate, if deemed appropriate.

Stopping—The occurrence of a serious adverse event (SAE) deemed related to the investigational product results in the suspension of any further dosing pending a safety review. A response of “0” on the MOAA/S (No response after painful trapezius squeeze) in a single participant also results in the suspension of any further dosing. The occurrence within the same cohort of two identical severe AEs deemed related to the investigational product results in the suspension of any further dosing pending a safety review. A response of “1” on the MOAA/S in two participants in a cohort also results in suspension of any further dosing. The occurrence within the same cohort of any three severe AEs deemed related to the investigational product results in the suspension of any further dosing. A response of “2” on the MOAA/S in three participants in a cohort also results in the suspension of any further dosing.

Sentinel Group—dosing of the full cohort occurs at least 24 hours following dosing of the sentinel participants assuming that no severe AEs are observed in either of the sentinel participants. If a severe AE or an MOAA/S score of 2 or lower is observed, dosing the remaining participants is suspended pending a safety review.

Re-starting—Depending upon the nature of the SEA or the severe AEs that cause dosing to be suspended, dosing is resumed at an intermediate level between the last successfully completed level and the dosing level at which the suspension occurred, after at least an assessment is made as to whether the informed consent firm needs to be revised and the patients reconsented. In the multiple ascending dose portion of the study, if a participant experiences a moderate or severe adverse event that is ongoing when the next dose is to be administered, further dosing may be suspended with the option to resume dosing once the event has resolved.

Adverse Events and Serious Adverse Events

AE Assessments—In the single ascending dose portion, participants are instructed to report AEs as they occur and will specifically be queried about the emergence of any AE prior to every post-dose PK blood sample draw. In the multiple dose portion, participants are instructed to report AEs as they occur and are specifically queried about the emergence of any AE prior to dosing and 4 times following dosing (with times determined based on single ascending dose results). Information that is collected includes event descriptions, times of onset, clinician's assessment of severity, relationship to investigational product, and date of resolution/stabilization of the event. All AEs occurring while on study are documented appropriately regardless of relationship and followed until resolution/stabilization is confirmed. If an adverse event is ongoing at discharge, the participant may be required to return to the clinic for follow-up or if a follow-up phone call is adequate. Any medical condition that is present prior to administration of the first dose of investigational product is considered part of medical history and not reported as an AE. If, however, the participant's condition deteriorates after the first dose of the investigational product, it will be recorded as an AE.

AE Definition and Reporting—An AE is an untoward medical occurrence in a participant administered a pharmaceutical product and that does not necessarily have a causal relationship with this treatment. An AE can therefore be an unfavorable and unintended sign (including an abnormal laboratory finding), symptom, or disease temporally associated with the use of an investigational product, whether or not considered related to the investigational product.

SAE definition and reporting—a serious adverse event or reaction is any untoward medical occurrence that, at any dose, has any of the following consequences: (i) results in death; (ii) is life-threatening; (iii) requires in-patient hospitalization or prolongation of existing hospitalization; (iv) results in persistence or significant disability/incapacity; (v) is a congenital anomaly/birth defect. All SAEs related to the investigational product or not, occurring during the study are reported. The SAE reporting period ends in discharge.

AE Classification—
Severity—The intensity of an AE is an estimate of the relative severity of the event. The following definitions are used to rate the severity of an AE.
   Mild: The symptom is barely noticeable to the participant and does not influence performance of daily activities. Treatment is not ordinarily indicated.
   Moderate: The symptom is sufficiently severe to make the participant uncomfortable, and performance of daily activities is influenced. Treatment may be necessary.
   Severe: The symptom causes severe discomfort, and daily activities are significantly impaired or prevented. Treatment may be necessary.
Relationship—Causality of the AE to the experimental treatment is established based on the participant's history, most recent physical examination findings, and concomitant medications. The following definitions are used to determine causality of an AE:
   No related: Temporal relationship of the onset of the AE, relative to the experimental treatment, is not reasonable, or another probably cause can readily explain the occurrence of the AE.
   Related: Temporal relationship of the onset of the AE, relative to the experimental treatment, is reasonable, follow a known response pattern to the treatment, and an alternative cause is unlikely.

Participant Duration

For both phases, participants are screened 1-29 days before admission. For the Single Ascending Dose portion, participants report ˜15 hours prior to dosing on Day 1 and remain for ˜48 hours following dosing for a total study duration of approximately 3 days. For the Multiple Ascending Dose portion,], participants report ˜15 hours prior to dosing on Day 1 and remain through ˜48 ours following dosing on Day 7 for a total study duration of approximately 9 days.

Discontinuation—The investigation product may be discontinued if any adverse event (AE), laboratory abnormality, or other medical condition or situation occurs such that continued participation in the study would not be in the best interest of the participant. Treatment discontinuation automatically leads to the participant's discontinuation.

Participant Discontinuation/Withdrawal from the Study—Participants have the right to withdraw from the study at any time for any reason without penalty. Participants may also be withdrawn from the study if in the best interest of the participant or if the participant is uncooperative or noncompliant. Should a participant decide to withdraw, all efforts are made to complete the end-of-study safety assessments. A complete final evaluation at the time of the participant's withdrawal is made with an explanation of why the participant is withdrawing from the study. If the reason for removal of a participant is an AE or an abnormal laboratory test result, the principal specific event of abnormal test result is recorded. Reasons for discontinuation include, but are not limited to, the following: (i) pregnancy; (ii) significant non-compliance; (iii) if any clinical adverse event (AE), laboratory abnormality, or other medical condition or situation occurs such that continued participation in the study would not be in the best interest of the participant; or (iv) if the participant meets an exclusion criterion (either newly developed or not previously recognized) that precludes further study participation.

### Discussion of Example 1

ATN-10155 is found to be safe and tolerably for administration to humans. Administration of ATN-10155 to study participants is found to result in minimal to no severe adverse events. Administration of ATN-10155 to study participants is found to result in minimal to no serious adverse events. The pharmacokinetic profile of ATN-10155 is found to be suitable for administration to human subjects.

ATN-10155 is found to be a potential treatment for a variety of CNS indications in humans, including seizure disorders, mood disorders, and related conditions.

### Example 2

The potency of the neurosteroids CV-10155, Sage-217, ganaxolone, and Praxis-114 on GABA_{A} receptors in preclinical studies was compared. Results are summarized in Table 3.

TABLE 3

| EC50 - potency (Emax - efficacy: | | | | | Praxis-114 |
|---|---|---|---|---|---|
| % of potentiation) | ETX-155* | | Sage-217* | Ganaxolone* | (S1 SEC report) |
| α1β2γ2 Synaptic receptors | 207 | (586%) | 189 (1000%) | 90 (745%) | 2241 (~1700%) |
| α4β3δ Extra synaptic receptors | 165 | (1530%) | 148 (3080%) | 213 (3320%) | 353 (~900%) |

*Data obtained by Synchropatch electrophysiology

The data show that CV-10155 is a dual potent synaptic and extra synaptic GABA_{A} positive allosteric modulator (PAM), with higher efficacy at extra synaptic channels. The dual potency of CV-10155 at synaptic and extra synaptic channels is similar to that of Sage-217 but different from Praxis-114, which is ˜7-fold more potent at extrasynaptic receptors. CV-10155 has 3-fold higher efficiency at extra synaptic vs. synaptic channels.

The effects of the neurosteroids CV-10155, Sage-217, and ganaxolone on various animal behaviors in preclinical studies were compared. Results are summarized in Table 4.

TABLE 4

| | | | Ganaxo- |
|---|---|---|---|
| | ETX-155 | SAGE-217 | lone |
| | (mg/kg) | (mg/kg) | (mg/kg) |
| MES - Seizure | 1 - 3 - - 10* | | 1 - 3 - 10 |
| PTZ - Seizure | 1* - 3* - - 10* | | 1 - 3 - 10 |
| GAERS - Absence seizure | | | |
| Marble burying - | 1 - 3 - 6* | | |
| Depression/Anxiety | | | |
| Forced swim test - | 1 - 3* - - 10* | 1 - 3* | |
| Depression | | | |
| Elevated Plus Maze - | 1 - 3 - 6* - 10* | 1 - 3* - 6* | |
| Anxiety | | | |
| Social interaction - | 1* - 3* - 6* | | |
| Anxiety/Depression | | | |
| EEG/Sleep | 1 - 3* - 6* - 10* | | |
| Sleep | 1 - 3* - 6* - 10* | | |

*Significantly efficacious dose.

The results show that CV-10155 is efficacious at 3 mg/kg in a broad range of preclinical models of depression, anxiety, seizure, and EEG. Overall, CV-10155 show efficacy in models of depression, anxiety, and seizure. The minimal effective dose (MED) is 1 mg/kg, robust efficacy was observed from 3 mg/kg onwards in most models.

The pharmacokinetic parameters of CV-10155 in preclinical studies on rodent models of seizure, anxiety, depression and EEG biomarker were analyzed. The results are summarized in Table 5.

TABLE 5

| | Minimal | | | | |
|---|---|---|---|---|---|
| | effective dose | | Effective Dose | | |
| ETX-155 | 1 mg/kg | 2 mg/kg | 3 mg/kg | 5 mg/kg | 10 mg/kg |
| Cₘₐₓ | 84.4-128 | 224 | 270-440 | 499 | 1165 |
| Tₘₐₓ | 1.5-2 | 1.7 | 1.25-2.50 | 0.83 | 1.25-2.25 |
| AUC_{0-inf} | 508-1301 | 1319 | 1378-3899 | 2610 | 6848-14667 |
| ½ life | 3.66-6.82 | nd | 2.84-4.96 | 4.19 | 3.23-4.80 |
| Oral BA | 58.6-92.4 | 47.2 | 53-92.3 | 58.1 | 79.7-100 |

1, 3 and 10 mg/kg data from study including male and female subjects
2 and 5 mg/kg data from study including only male subjects

The results show that preclinical minimal efficacious dose is 1 mg/kg, and robust efficacy observed from 3 mg/kg onwards across several rodent models of seizure, anxiety, depression and EEG biomarker.

### Example 3

The pharmacokinetic properties of CV-10155 in humans were analyzed. Subjects were given a single oral dose of CV-10155, and plasma concentrations were measured at various time points. Various dosages were given to fasting subjects, and 30 mg dose was given to a subjects in a fed state.

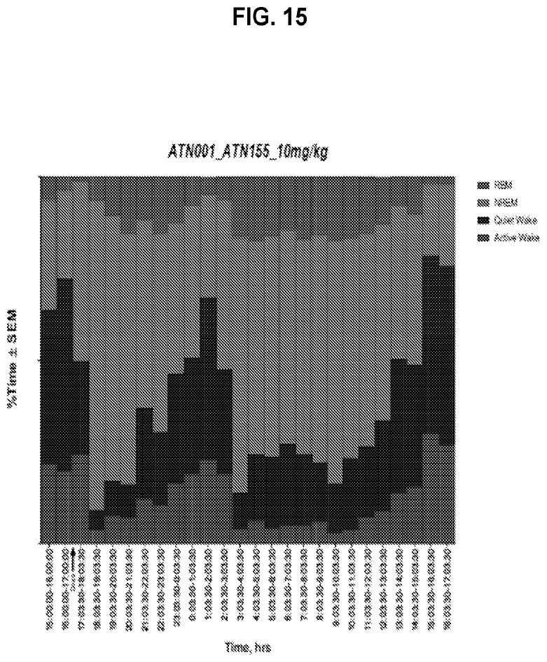

FIG. 15 is a graph of the average plasma concentration of CV-10155 following oral administration to humans. Purple circles represent 5 mg, fasted; green diamonds represent 15 mg, fasted; red squares represent 30 mg fasted; blue triangles represent 30 mg, fed; purple crosses represent 60 mg, fasted; teal 5-pointed stars represent 90 mg, fasted; olive 6-pointed stars represent 135 mg, fasted; and brown 10-pointed stars represent 200 mg fasted.

FIG. 16 is a graph of the average plasma concentration of CV-10155 following oral administration of a 30 mg dose to humans. Blue triangles represent fasted; and red circles represent fed.

FIG. 17 is a graph showing the ratios of Cₘₐₓ and AUC between fed and fasted subjects.

CV-10155 displays a dose-proportional increase for AUC and Cₘₐₓ across 5-200 mg dose range with small/moderate inter-subject variability (CV≤30%). The Tₘₐₓ is ˜2-4 hours, and the half-life is ˜24-26 hours. No dose-limiting adverse events were observed over the range of 5-135 mg; ataxia, tremor and tachycardia were observed at 200 mg. Dizziness and somnolence, which were mild to moderate, were the most common adverse events and were only observed at the highest doses.

Taken together, the results show that food consumption has no clinically meaningful effect on the absorption of CV-10155 following oral administration of the drug. These findings support the oral administration of CV-10155 in a dosing regimen that is agnostic regarding food consumption. In particular, the results indicate that CV-10155 is suitable for oral administration during fasting periods and need not be taken with food.

### Example 4

The effects of CV-10155 in relation to administration at different times of the day and states of feeding were analyzed in humans. Subjects were given 60 mg CV-10155 orally once per day for seven consecutive days, and plasma concentrations were measured at various time points. Doses were administered either in the morning to fasted subjects or in the evening to fed subjects.

FIG. 18 is a graph of the average plasma concentration of CV-10155 following oral administration to humans. Red circles represent morning, fasted administration; and blue triangles represent evening, fed administration.

Adverse events observed following administration of a single dose of CV-10155 in the evening in a fed state are summarized on Table 6.

TABLE 6

| | | | Onset | | |
|---|---|---|---|---|---|
| | | Dosing | from dose | Duration | |
| Subject | AE | day | (h) | (h) | Severity |
| a | Somnolence | 1 | 00:45 | 10.62 | Mild |
| v | Back pain | 4 | 00:15 | 39.25 | Mild |
| l | Headache | 1 | pre-dose | 13.25 | Mild |
| o | Abdominal | 3 | 17:20 | 06.50 | Mild |
| | distension | | | | |
| p | Dizziness | 2 | 01:10 | 20.50 | Mild |
| p | Dizziness | 7 | 01:10 | 13.00 | Mild |
| p | Headache | 8 | 21:40 | 14.50 | Moderate |

A comparison of adverse events observed following administration to fasted subjects in the morning and to fed subjects in the evening is provided in Table 7.

TABLE 7

| | | | N = 9 | N = 9 |
|---|---|---|---|---|
| | | | 60 mg, | 60 mg, |
| | | | morning, | evening, |
| | | N = 3 | fasted | fed |
| | AE | Placebo | state | state |
| AE SOC | description | # | # | # |
| Gastrointestinal disorders | Bloating | — | — | 1 |
| General disorders and | Tiredness | — | 3 | — |
| administration site | Feeling tired | — | 2 | — |
| conditions | | | | |
| Musculoskeletal and | Back pain | — | — | 1 |
| connective tissue | | | | |
| disorders | | | | |
| Nervous system disorders | Sleepiness | 2 | 9 | — |
| | Headache | 2 | 2 | — |
| | Dizziness | — | 1 | 1 |
| | Feeling | — | 1 | — |
| | sleepy | | | |
| Nervous system disorders | Sleepiness | — | — | 1 |
| | Headache | — | — | 2 |
| Nervous system disorders | Sleepiness | 1 | — | 1 |

The results show no significant difference in absorption of CV-10155 following oral administration between subjects that received it in the morning in a fasted state and subjects that received it in the evening in a fed state. In addition, oral administration of CV-10155 in the evening to fed subjects does not produce adverse effects that interfere with sleeping.

Taken together, the results indicate that oral formulations of CV-10155 are suitable for a dosing regimen in which the drug is provided in the evening and/or prior to an extended period of sleep.

### Example 5

The pharmacokinetic properties of CV-10155 obtained from preclinical studies and from studies on humans were compared to determine comparable dosing levels between animals and humans.

FIG. 19 is a graph of Cₘₐₓ ranges from studies on rats, dogs, and humans. Blue open box represents day 1 data from humans given oral dose in the morning in a fasted state; green open box represents day 7 data from humans given oral dose in the morning in a fasted state; red open box represents day 1 data from humans given oral dose in the evening in a fed state; purple open box represents day 7 data from humans given oral dose in the evening in a fed state; solid orange line indicates levels associated with ataxia in humans; solid dark green box indicates levels associated with robust efficacy in preclinical studies, and solid light green box indicates levels associated with the minimum effective dose in preclinical studies. The ratios of Cₘₐₓ/trough on day 7 from human studies are as follows: for 60 mg CV-10155 administered daily in the morning in a fasted state, 6.09; for 60 mg CV-10155 administered daily in the evening in a fed state, 3.47; and for 30 mg Sage-2017 administered daily in the evening in a fed state, 5.88.

FIG. 20 is a graph of AUC₀₋₂₄ ranges from studies on rats, dogs, and humans. Blue open box represents day 1 data from humans given oral dose in the morning in a fasted state; green open box represents day 7 data from humans given oral dose in the morning in a fasted state; red open box represents day 1 data from humans given oral dose in the evening in a fed state; purple open box represents day 7 data from humans given oral dose in the evening in a fed state; solid orange line indicates levels associated with ataxia in humans; solid dark green box indicates levels associated with robust efficacy in preclinical studies, and solid light green box indicates levels associated with the minimum effective dose in preclinical studies.

Taken together, the results show that the values of pharmacokinetic parameters resulting from oral administration of 60 mg CV-10155 in humans are similar to those observed when the drug is provided in efficacious doses to dogs and rats in preclinical animal models.

### Example 6

The tolerability of the neurosteroids CV-10155, Sage-217, and Praxis-114 in human subjects were compared. The results from subjects that received 45 mg Praxis-114 in the evening, 60 mg Praxis-114 in the evening, 80 mg Praxis-114 in the evening, or 60 mg CV-10155 in the evening are provided in Table 8, and the results form subjects that received 30 mg Sage-217 or 20 mg Sage-217 are provided in Table 9.

TABLE 8

| | Praxis-114 MDD | Praxis-114 MDD | Praxis-114 MDD | ETX-155 HV |
|---|---|---|---|---|
| | 45 mg | 60 mg | 80 mg | 60 mg |
| | evening dosing | evening dosing | evening dosing | Evening dosing |
| | AEs % of subjects | AEs % of subjects | AEs % of subjects | AEs % of subjects |
| | (n = 13) | (n = 13) | (n = 7) | (n = 12) |
| Somnolence | 15.4% | 53.8% | 42.9% | 8.3% |
| Fatigue | 23.1% | — | — | — |
| Headache | 53.8% | 46.2% | 42.9% | 8.3% |
| Dizziness | — | 30.8% | 57.1% | 16.7% |
| Feeling drunk | — | 23.1% | 28.6% | — |

TABLE 9

| | Sage-217 30 mg | | Sage-217 20 mg | | Placebo | |
|---|---|---|---|---|---|---|
| | (n-192) | | (n-188) | | (n = 190) | |
| Any - n (%) | 104 | (54.2) | 94 | (50.0) | 93 | (48.9) |
| Headache | 12 | (6.3) | 21 | (11.2) | 14 | (7.4) |
| Dizziness | 11 | (5.7) | 14 | (7.4) | 7 | (3.7) |
| Somnolence | 13 | (6.8) | 11 | (5.9) | 8 | (4.2) |
| Fatigue | 13 | (6.8) | 3 | (1.6) | 5 | (2.6) |
| Diarrhea | 12 | (6.3) | 11 | (5.9) | 10 | (5.3) |
| Sedation | 9 | (4.7) | 11 | (5.9) | 6 | (3.2) |
| Nausea | 7 | (3.6) | 10 | (5.3) | 9 | (4.7) |

The results show that CNS adverse events, such as somnolence, fatigue, dizziness, are common to all three compounds. Ataxia is the dose-limiting adverse event for CV-10155. The data suggest that at 60 mg dosed in the evening, CV-10155 has a favorable tolerability profile compared to PRAX-114 and one comparable to that of Sage-217.

### Example 7

The effect of CV-10155 on various sleep states was analyzed in rats. Rats were given various doses of CV-10155, and sleep states were analyzed by electroencephalogram (EEG).

FIG. 21 is a hypnogram showing the percentage of time spent in different sleep states by rats given drug vehicle. Purple bars represent REM sleep; green bars represent non-REM sleep; blue bars represent quiet waking; and red bars represent active waking.

FIG. 22 is a hypnogram showing the percentage of time spent in different sleep states by rats given 1 mg/kg CV-10155. Purple bars represent REM sleep; green bars represent non-REM sleep; blue bars represent quiet waking; and red bars represent active waking.

FIG. 23 is a hypnogram showing the percentage of time spent in different sleep states by rats given 3 mg/kg CV-10155. Purple bars represent REM sleep; green bars represent non-REM sleep; blue bars represent quiet waking; and red bars represent active waking.

FIG. 24 is a hypnogram showing the percentage of time spent in different sleep states by rats given 6 mg/kg CV-10155. Purple bars represent REM sleep; green bars represent non-REM sleep; blue bars represent quiet waking; and red bars represent active waking.

FIG. 25 is a hypnogram showing the percentage of time spent in different sleep states by rats given 10 mg/kg CV-10155. Purple bars represent REM sleep; green bars represent non-REM sleep; blue bars represent quiet waking; and red bars represent active waking.

The results show that CV-10155 displays dose-dependent target engagement within well-tolerated and efficacious dose ranges. In addition, CV-10155 produces an increase in non-REM sleep at doses of 3 mg/kg and higher.

These findings indicate that CV-10155 improves sleep quality in animal models and suggest that CV-10155 may be useful to treat sleep disorders in human.

### Example 8

The pharmacokinetic properties of CV-10155 and Sage-217 in humans were compared. The day 7 from subjects given either 60 mg CV-10155 in the morning in a fasted state or 30 mg Sage-217 were analyzed. Results from multiple ascending dose studies are shown in Table 10, and results from single ascending dose studies are shown in Table 11.

TABLE 10

| | Cₘₐₓ | Cₘᵢₙ | | Tₘₐₓ | T_{1/2} | AUC_{0-inf} | Racc | Racc |
|---|---|---|---|---|---|---|---|---|
| MAD | (ng/ml) | (ng/ml) | Ratio | (h) | (h) | (h*ng/ml) | Cₘₐₓ | AUC |
| ETX-155 60 mg | 252.56 | 41.5 | 6.07 | 3 | 34.48 | 2899.9 | 1.78 | 1.84 |
| day 7 MAD | | | | | | | | |
| Morning dosing | | | | | | | | |
| SAGE-217 30 mg | 115.2 | 12.46 | 9.24 | 1 | 15.27 | 833.2 | 0.943 | 1.298 |
| day 7 MAD (MTD) | | | | | | | | |
| Morning dosing | | | | | | | | |

TABLE 11

| | Cₘₐₓ | Tₘₐₓ | T_{1/2} | AUC_{0-inf} |
|---|---|---|---|---|
| SAD | (ng/ml) | (h) | (h) | (h*ng/ml) |
| ETX-155 135 mg SAD MTD | 332.8 | 2.83 | 25.0 | 3661 |
| SAGE-217 55 mg SAD MTD | 149.9 | 1.24 | 17.02 | 1633 |

Taken together, the results show that administration of 60 mg doses of CV-10155 achieves higher exposure levels in humans than does administration of 30 mg doses of Sage-217.

### Example 9

Further pharmacokinetic analysis on the plasma concentrations of the Phase 1 human dosing exposure studies indicates the following:
ETX-020155 plasma concentration reaches Tmax approximately 3-4 hours after oral administration and after that declining with a terminal half-life of approximately 40 hours both after single and repeat administration.
Visual inspection of the mean concentrations of ETX-020155 data indicates steady state was achieved by the eighth of daily dose of ETX-020155 and that accumulation ratio based on AUC was approximately 2 with a once daily administration.
ETX-020155 elimination in urine is estimated to be <1% of the dose indicating metabolism as the likely main pathway for the elimination.
Repaglinide exposure was not to be significantly affected by repeat administration of ETX-020155 with Cmax and AUC approximately 15% higher after 14 day ETX-020155 repeat doses indicating no or minimal DDI inhibitory effect at CYP2C8 level.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of treating a central nervous system related disorder in a human subject, the method comprising providing a pharmaceutical composition to a human subject having a central nervous system disorder, wherein the pharmaceutical composition is an oral formulation that comprises a compound of Formula (I): 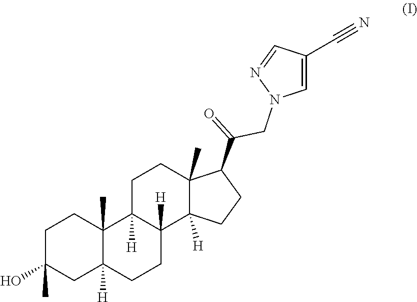
at a dose between 5 mg and 90 mg.

2. The method of claim 1, wherein the pharmaceutical composition is provided at a dose of 5 mg, 15 mg, 30 mg, 60 mg, or 90 mg.

3. The method of claim 2, wherein the pharmaceutical composition is provided once daily.

4. The method of claim 3, wherein the pharmaceutical composition is provided once daily for seven days.

5. The method of claim 2, wherein the pharmaceutical composition is provided to the subject in the morning.

6. The method of claim 2, wherein the pharmaceutical composition is provided to the subject in the evening.

7. The method of claim 1, wherein the disorder is a GABA_{A} receptor mediated disorder.

8. The method of claim 7, where the disorder is selected from the group consisting of seizure disorders and mood disorder.

9. The method of claim 1, wherein the method results in minimal severe adverse reactions related to administration of the pharmaceutical composition.

10. The method of claim 9, wherein the method results in no severe adverse reactions related to administration of the pharmaceutical composition.

11. The method of claim 1, wherein the method results in no serious adverse reactions related to administration of the pharmaceutical composition.

12. The method of claim 2, wherein the pharmaceutical composition is provided twice daily.

13. The method of claim 12, wherein the pharmaceutical composition is provided once in a morning and once in an evening.
